(19) United States
(12) Patent Application Publication
(10) Pub. No.: US 2010/0297116 A1
(43) Pub. Date: Nov. 25, 2010

(54) USES OF A GLYCOPROTEIN VI (GPVI) INHIBITOR

(76) Inventors: Yongge Liu, Germantown, MD (US); Narendra N. Tandon, Gaithersburg, MD (US); Hisao Takizawa, Matsushige (JP); Junichi Kambayashi, Potomac, MD (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/740,620

(22) PCT Filed: Oct. 29, 2008
(86) PCT No.: PCT/US08/12311

§ 371 Date: Aug. 16, 2010

Related U.S. Application Data
(60) Provisional application No. 60/984,334, filed on Oct. 31, 2007.

(51) Int. Cl. A61K 39/395; C07K 16/18

(52) U.S. Cl. 424/133.1; 424/130.1; 530/387.1; 530/387.3

(57) ABSTRACT

The present invention describes a method for reducing reperfusion injury and/or infarction by using an inhibitor of platelet GPVI. This method may be used to treat patients during or after a heart attack or elective cardiac surgery.

### RELATED APPLICATIONS

This application claims the benefit of priority of U.S. Application No. 60/984,334, “Uses of a Glycoprotein VI (GPVI) Inhibitor,” filed Oct. 31, 2007, which is incorporated by reference in its entirety.

### FIELD OF THE INVENTION

The present invention relates to a method of inhibiting reperfusion injury and/or infarction using inhibitors of platelet membrane glycoprotein VI (GPVI), including antibodies, protein fragments, and small molecular compounds.

### BACKGROUND

A heart attack occurs when a coronary artery supplying blood to the heart becomes blocked. Blockage usually occurs due to the narrowing and closing of the artery as a consequence of atherosclerosis and thrombus formation. The lack of blood supply is referred to as ischemia. Heart muscle can only tolerate a short period of oxygen starvation and will infarct in less than 20-120 min. Because heart muscle cells are largely terminally differentiated, the heart has very limited ability to regenerate. Patients who have had a heart attack will carry a heart with infarcted tissue for the rest of their lives. Because infarcted heart muscle has reduced ability to pump blood, these patients will have reduced ability to maintain blood supply to the body. After a heart attack, congestive heart failure may follow and patients may also experience recurrent heart attacks. Patients with heart failure have reduced mobility, decreased quality of life, and shortened life span.

According to the most recent statistics from the American Heart Association, there are 1.2 million heart attacks yearly in the U.S. alone (Thom et al., Circulation, 113:e85-151, 2006). There are currently 5 million people with heart failure and 550,000 new cases each year in the U.S. (Thom et al., Circulation, 113:e85-151, 2006).

A blocked coronary artery may be reopened with angioplasty and/or thrombolytic therapy, resulting in reperfusion of the previously ischemic muscle. While reperfusion is essential to salvage the ischemic muscle, reperfusion itself may paradoxically cause additional damage to the muscle. Ideally, treatment for a heart attack would involve minimizing myocardial infarction during the attack. However, because it is usually difficult to predict the occurrence of a heart attack, prophylactic treatment is unlikely. Thus, angioplasty and/or thrombolytic therapy combined with treatment that reduces reperfusion injury (for example, given in the ambulance or the emergency room) may be more practical. The treatment that reduces reperfusion injury will likely improve recovery from a heart attack/ischemia, and limit the possibility of developing heart failure. Treatments that reduce myocardial infarction are anticipated to be life-saving and can reduce hospitalization time, enhance quality of life, and reduce overall health care costs of high risk patients.

Unfortunately, no such treatment is currently available. Various treatments have been attempted and all appear to have failed (see a review by Downey and Cohen, Prog Cardiovasc Dis, 48:363-371, 2006). Antithrombotic interventions, such as aspirin, clopidogrel, and ReoPro®, are currently recommended to prevent occlusion/reocclusion of the coronary artery. However, they do not provide direct protection against reperfusion injury. Aspirin may in fact interfere with some of the endogenous cardioprotective pathways, and may increase infarction (Gross et al., J Pharmacol Exp Ther, 310:185-191, 2004).

### SUMMARY OF THE INVENTION

The present invention provides a method for inhibiting reperfusion injury and/or infarction in a patient by administering an inhibitor of platelet glycoprotein VI (GPVI), a major collagen receptor present on the platelet surface. The present invention also provides a use of such an inhibitor for the manufacture of a medicament for the treatment of reperfusion injury and/or infarction.

GPVI is exclusively expressed on platelets and megakaryocytes and binds to collagen, which is one of the most thrombogenic matrix proteins underneath the vascular endothelium. Rupture of the atherosclerotic plaques, ischemia, and reperfusion injury may expose collagen to blood elements, including platelets. The binding of platelet GPVI to collagen plays a pivotal role in the adhesion of platelets at the site of injured vasculature and subsequent platelet activation and aggregation. An inhibitor of platelet GPVI blocks the interaction between platelet GPVI and collagen found in the vessel wall. While GPVI inhibition has been previously shown to reduce platelet activation, the present invention shows that GPVI inhibition also unexpectedly provides a direct cardioprotective effect and is useful in inhibiting reperfusion injury and/or infarction.

The inhibitor of platelet GPVI may be an antibody, protein fragment, or a small molecular compound. The antibody includes, but is not limited to, a monoclonal anti-GPVI antibody. The monoclonal antibody includes an active antibody fragment. An active antibody fragment may be a chemically, enzymatically, or recombinantly produced Fab fragment, F(ab)₂ fragment, or peptide comprising at least one complementarity determining region (CDR) specific for a GPVI polypeptide, peptide, or naturally-occurring variant thereof. Exemplary antibodies include murine monoclonal antibodies OM1, OM2, OM3, and OM4 and their humanized version or their active fragments. The peptide fragment includes, but is not limited to, collagen-binding domains of GPVI and soluble GPVI.

### BRIEF DESCRIPTION OF THE DRAWINGS

FIG. 1 shows a comparison of myocardial infarct size in wild type and GPVI knockout mice. Myocardial infarction was significantly smaller in GPVI knockout mice compared to wild type mice after 30 min ischemia and 24 hr reperfusion. Each open circle represents the infarct size from an individual mouse, and the closed circle represents the group mean value±SD. Data were analyzed with the t-test and p<0.05 is considered statistically significant.

FIG. 2a shows a comparison of P-selectin expression in the myocardium of wild type and GPVI-knockout mice after ischemia and reperfusion. Representative fluorescent images from the endocardium and midmyocardium are shown. P-selectin expression as shown in bright green color (or bright whitish color in a black-and-white version of the figure) was reduced in myocardium from GPVI-knockout mice (dim background fluorescence was due to autofluorescence of the myocardium). Similar results were obtained in 5 hearts from wild type mice and 5 hearts from GPVI-knockout mice, respectively. FIG. 2b shows a quantitation of the areas with high P-selectin expression. GPVI-knockout (KO) mice have significantly lower levels of P-selectin than wildtype (WT) mice (n=5), indicating that GPVI plays an important role in inducing platelet activation and aggregation in the myocardium.

FIG. 3 demonstrates the exposure of collagen in the heart of a wildtype mouse due to reperfusion after ischemia. The left panel shows a representative section from a heart subjected to 30 min ischemia followed by 15 min reperfusion. Bright green color (or bright whitish color in a black-and-white version of the figure) represents exposed collagen (dim background fluorescence was due to autofluorescence of the myocardium). Similar results were obtained from 3 additional animals. The right panel shows a representative section from a heart that was exposed to 30 min ischemia but without subsequent reperfusion. No green fluorescence (or no bright whitish color in a black-and-white version of the figure) was apparent, indicating that no collagen was exposed. Similar results were obtained from 2 additional animals. Together, these data show that endothelial injury occurs during reperfusion.

FIG. 4a demonstrates the infarction-reducing effect of the anti-GPVI antibody OM2 in monkeys. The figure shows a scatter plot of risk zone vs. infarct area with a regression line drawn for each of the indicated treatment groups. The infarction in control monkeys was linearly related to the size of the risk zone. Monkeys with either single or double dose treatment with OM2 had reduced myocardial infarction since all data points were below the regression line of the control (p<0.05). Furthermore, the reduction was similar in monkeys treated with a single or a double dose, suggesting that the protection by OM2 occurred during the reperfusion period. Infarct data were analyzed by analysis of variance (ANOVA). FIG. 4b demonstrates the inhibition of platelet aggregation in blood of monkeys by OM2. Blood samples were withdrawn from monkeys before (pre-dosing) and after (4 hrs post-dosing) OM2 administration (2 mg/kg). Collagen-induced platelet aggregation was determined in an ex vivo assay using a whole blood aggregometer. FIG. 4b shows representative measurements of collagen-induced platelet aggregation. Collagen-induced platelet aggregation was completely inhibited in the whole blood of animals that had received OM2.

### DESCRIPTION OF THE EMBODIMENTS

An “infarction” generally refers to necrosis of tissue due to upstream obstruction of its arterial blood supply. The lack of oxygenated blood starves the cell to death. An infarction can affect any organ, but occurs more often and faster (<20-120 minutes) in tissue with high energy demand and metabolic activity such as the heart.

The term “myocardial infarction” herein refers to myocardial necrosis usually resulting from abrupt reduction in coronary blood flow to a segment of the myocardium. The myocardium can only sustain a very short period of ischemia (<5 min) without suffering an injury. Reversible injury generally occurs between 5 to 20 min if blood flow does not resume. A longer period of ischemia usually results in permanent injury, i.e., cell death/necrosis/infarction. Because the myocardium has very limited ability to regenerate, the loss of muscle may be permanent. “Endothelial dysfunction” refers to endothelium necrosis or loss of normal function resulting from ischemia and reperfusion.

“Reperfusion injury” refers to damage to tissue caused when blood supply returns to the tissue after a period of ischemia. The absence of oxygen and nutrients from blood creates a condition in which the restoration of circulation results in inflammation and oxidative damage through the induction of oxidative stress rather than restoration of normal function. “Myocardial reperfusion injury” refers to reperfusion injury occurring in the myocardium, and “endothelial reperfusion injury” refers to reperfusion injury occurring in the endothelium.

“Patient” herein refers to any person or non-human animal in need of treatment to reduce the incidence, likelihood, or degree of infarction and/or reperfusion injury. “Patient” also includes subjects that have suffered or are at risk for a heart attack, including, but not limited to those that have been diagnosed with cardiovascular disorders such as coronary artery disease (CAD), systemic hypertension, bicuspid aortic valve, hypertrophic cardiomyopathy, or mitral valve prolapse; those that experience or have experienced a heart attack and/or heart failure (including congestive heart failure (CHF)); and those that are subjected to elective cardiac surgery that requires temporary blocking of coronary artery blood flow, for example, during cardiac by-pass surgery. Non-human animals to be treated include all domesticated and feral vertebrates, including, but not limited to mice, rats, rabbits, fish, birds, hamsters, dogs, cats, swine, sheep, horses, cattle, and non-human primates.

The term “inhibit” refers to a decrease or cessation of any phenotypic characteristic or to the decrease or cessation in the incidence, degree, or likelihood of that characteristic. Thus, “inhibiting reperfusion injury and/or infarction” refers to a measurable decrease or cessation in reperfusion injury and/or infarction.

“Inhibitor of platelet GPVI” refers to any antibody, protein fragment, or small molecular compound capable of inhibiting the function of platelet GPVI. The function of platelet GPVI includes interaction of platelet GPVI with collagen found, for example, in the vascular wall. Other functions include collagen-induced platelet aggregation, platelet adhesion to immobilized collagen, collagen-induced ATP secretion, and collagen-induced thromboxane A₂ formation.

The term “antibody” is well-known in the art and includes monoclonal antibodies. The monoclonal antibodies of the invention include active antibody fragments, such as chemically, enzymatically, or recombinantly produced Fab fragments, F(ab)₂ fragments, or peptide fragments comprising at least one complementarity determining region (CDR) specific for a GPVI polypeptide, peptide, or naturally-occurring variant thereof. Anti-GPVI antibodies are “specifically binding” if they bind a GPVI polypeptide, peptide, or naturally-occurring variant thereof, with a dissociation constant (Kd) equal to or lower than 10⁻⁷ M. In another embodiment of the invention, the anti-GPVI antibodies specifically bind to a GPVI polypeptide, peptide, or naturally-occurring variant thereof, at a Kd of equal to or lower than 10⁻⁸ M. In a further embodiment, the anti-GPVI antibodies of the invention specifically bind to a GPVI polypeptide, peptide, or naturally-occurring variant thereof, at a Kd of equal to or lower than 10⁻⁹ M. Affinities of binding partners or antibodies may be readily determined using conventional techniques, for example, by measuring the saturation binding isotherms of ¹²⁵I-labeled IgG or its fragments, or by homologous displacement of ¹²⁵I-labeled IgG by unlabeled IgG using nonlinear-regression analysis as described by Motulsky, in Analyzing Data with GraphPad Prism (1999), GraphPad Software Inc., San Diego, Calif. Other techniques are known in the art, for example, those described by Scatchard et al., Ann. NY Acad. Sci., 51:660 (1949).

U.S. Patent Application Publication No. 2007/0207155 describes in detail the production of monoclonal antibodies and their humanization. U.S. Patent Application Publication No. 2007/0207155 also describes monoclonal antibodies OM1, OM2, OM3, and OM4 having the above described binding properties, as well as peptide fragments comprising at least one complementarity determining region (CDR) specific for a GPVI polypeptide, peptide, or naturally-occurring variant thereof. Furthermore, GPVI polypeptides, peptides, or naturally-occurring variants thereof, are described in U.S. Pat. No. 6,998,469 and in U.S. Patent Application Publication No. 2007/0207155, both of which are incorporated herein by reference in their entirety.

“Small molecular compound” refers to an organic, non-protein compound up to 1500 Da in size. A small molecular compound may be synthetic or derived from natural product extracts. A key structural feature is often a rigid, multi-ring core structure that reduces entropic cost paid on binding of the small molecule to a protein. The small molecular compounds of the invention inhibit the function of platelet GPVI, including but not limited to, the interaction of platelet GPVI with collagen.

As discussed above, “peptide fragment” includes peptide fragments comprising at least one CDR specific for a GPVI polypeptide, peptide, or naturally-occurring variant thereof, examples of which are disclosed in U.S. Patent Application Publication No. 2007/0207155. Other peptide fragments may include collagen binding domains of GPVI. The full GPVI sequence is disclosed in Clemetson et al., J. Biol. Chem. 274:29019-24 (1999); WO 00/68377; Jandrot-Perrus et al., Blood 96:1798-807 (2000); and Ezumi et al., Biochem Biophys Res Commun. 277:27-36 (2000), and the primary collagen binding surface on GPVI has been mapped. O'Connor et al., J. Biol. Chem. 281(44):33505-10 (2006); Dumont et al., J. Mol. Biol. 361(5):877-87 (2006); Horii et al., Blood 108(3):936-42 (2006); O'Connor et al., J Thromb Haemost. 4(4):869-73 (2006). In addition, soluble GPVI (sGPVI), which comprises the extracellular domain of GPVI, has been shown to inhibit the binding of GPVI to collagen, thereby inhibiting collagen-induced platelet aggregation. Jandrot-Perrus et al., Blood 96:1798-807 (2000).

The treatment of a patient comprises the administration of a pharmaceutically effective amount of an inhibitor of platelet GPVI. One of ordinary skill in the art may empirically determine the optimum dosage and dosage schedule for administering these inhibitors. Nevertheless, a pharmaceutically effective amount is an amount which provides an inhibition of reperfusion injury, infarction, or ischemic events in a patient.

A pharmaceutically effective amount may be administered as a single dose or as multiple doses over the course of treatment. The inhibitors of the invention may be administered by any method familiar to those of ordinary skill in the art, for example, intravenous (IV) administration by bolus injection, continuous infusion, or intermittent infusion. In alternative embodiments, the inhibitors may be administered intraperitoneally (IP), intracorporeally, intra-articularly, intraventricularly, intrathecally, intramuscularly (IM), subcutaneously, topically, tonsillarly, mucosally, intranasally, transdermally, intravaginally, orally, or by inhalation.

The present invention is illustrated by the following Examples, which are not intended to be limiting in any way.

### Example 1

### Deletion of GPVI is Cardioprotective in Mice

Age-matched wild type and GPVI knockout mice were anesthetized with 1-1.5% isoflurane and intubated via an endotracheal tube, and attached to a pressure controlled respirator. The animals were ventilated with room air supplemented with 100% oxygen (4:1 volume ratio). Before starting surgery, mice were given gentamicin (0.7 mg/kg IM). Body temperature was carefully monitored with a rectal probe connected to a digital thermometer and was maintained between 37 to 37.5° C. throughout the experiment using a heating pad and a heat lamp. In preliminary studies, a catheter was inserted into the carotid artery for measurement of blood pressure and analysis of blood gases. This was to insure that mice could maintain physiological hemodynamics using these experimental procedures.

With the aid of a dissecting microscope, the chest was opened through a left thoracotomy. An 8-0 nylon suture (Ethicon, Inc. Johnson & Johnson Co. Somerville, N.J.) was passed with a tapered needle under the left anterior descending coronary artery 2-3 mm from the tip of the left auricle, and the ends of the suture were passed through a plastic tube. Coronary occlusion was induced by pulling the suture against the tube. Successful performance of coronary occlusion and reperfusion was verified by visual inspection (i.e., by noting the development of a pale color in the distal myocardium upon pulling the suture and the return of a bright red color due to hyperemia after deflation) during ischemia and its resolution after reperfusion. The ischemia lasted 30 min. After the release of occlusion, the chest was closed in layers with sutures. A dose of Ketofen (2.5 mg/kg, IM) was injected. Upon gaining spontaneous respiration, the mice were removed from the ventilator, and placed into a temperature/humidity controlled unit with oxygen-enriched air. After the mice gained normal postural capabilities, they were then returned to cages for 24 hours.

At the conclusion of the study (the second day), the mice were given heparin (1 U/g IP) and were subsequently anesthetized with sodium pentobarbital (100 mg/kg IP). The heart was excised and perfused with Krebs-Henseleit solution through an aortic cannula (23-gauge needle) using a Langendorf apparatus. To delineate the occluded and then reperfused region (the region at risk), the coronary artery was tied at the site of the previous occlusion and the aortic root was perfused with a 1% solution of fluorescent particles (1-10 μm in diameter, Duke Scientific, Palo Alto, Calif.) in normal saline (1 mL over 3 min). As a result of this procedure, the portion of the left ventricle (LV) supplied by the previously occluded coronary artery (region at risk) was identified by the absence of fluorescence under a UV light, whereas the rest of the LV was stained dark blue. The heart was frozen for 20 min, and subsequently cut into 5-7 transverse slices. To delineate infarcted from viable myocardium, the heart slices were incubated in 1% solution of triphenyltetrazolium chloride (TTC) in phosphate buffer (pH 7.4, 37° C.) for 20 min. The slices were then fixed in 10% neutral buffered formaldehyde and, 24 h later, photographed. The borders of the infarcted, ischemic-reperfused (risk area), and nonischemic regions were traced. The corresponding areas were measured by computerized planimetry and from these measurements infarct size was calculated as a percentage of the risk area.

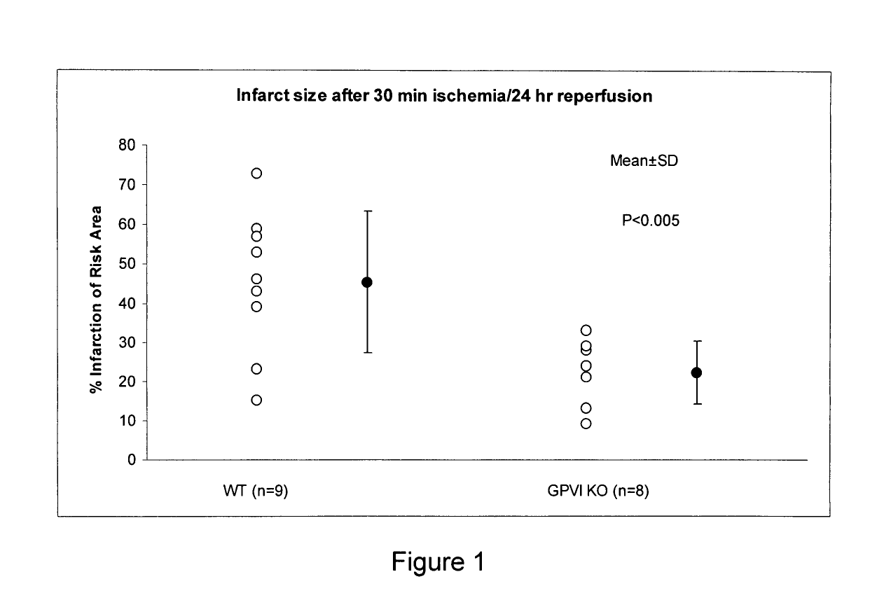

Risk areas were similar in size between wild type and GPVI knockout mice (0.020±0.004 cm³ and 0.022±0.005 cm³, respectively). Infarcted areas (infarct size) in wild type mice averaged 45±18% of the risk areas. The infarcted areas (infarct size) were significantly smaller in GPVI-knockout mice, averaging 22±8% of the risk areas. These data are summarized in FIG. 1.

### Example 2

### Reduction of P-Selectin Expression in Myocardium of GPVI Knockout Mice

The activation of platelets was determined by the expression of P-selectin, which is stored in platelet α-granules and can rapidly translocate to the platelet surface upon activation. P-selectin expression was examined using immunohistology.

In vivo cardiac ischemia/reperfusion in mice: Mouse heart ischemia and reperfusion was performed as described in EXAMPLE 1. GPVI knockout and wild type mice received 30 min of left anterior descending coronary artery (LAD) occlusion followed by 15 min of reperfusion. After 15 min reperfusion of LAD, the hearts were harvested and washed using DPBS, then cut into two short-axis parts and immediately placed in 4% paraformaldehyde and 0.1 M phosphate buffer to fix the tissues. After 2 hours, tissues were transferred to 25% sucrose overnight.

Immunofluorescence detection of P-selectin: On the 2nd day, heart tissue was cut into 20 μm cross-sections and allowed to dry for about 30 minutes. Sections on each slide were encircled with a PAP pen ring and let to dry for about 10 minutes. Slides were rinsed in 0.01 M PBS-0.1% Triton (PBST), and incubated with normal donkey serum (10% NDS, PBST) for 30-60 minutes at room temperature. P-selectin was detected using a rabbit anti-mouse P-selectin polyclonal antibody (Chemicon International), and visualized with a FITC-anti-rabbit IgG (Jackson ImmunoResearch lab).

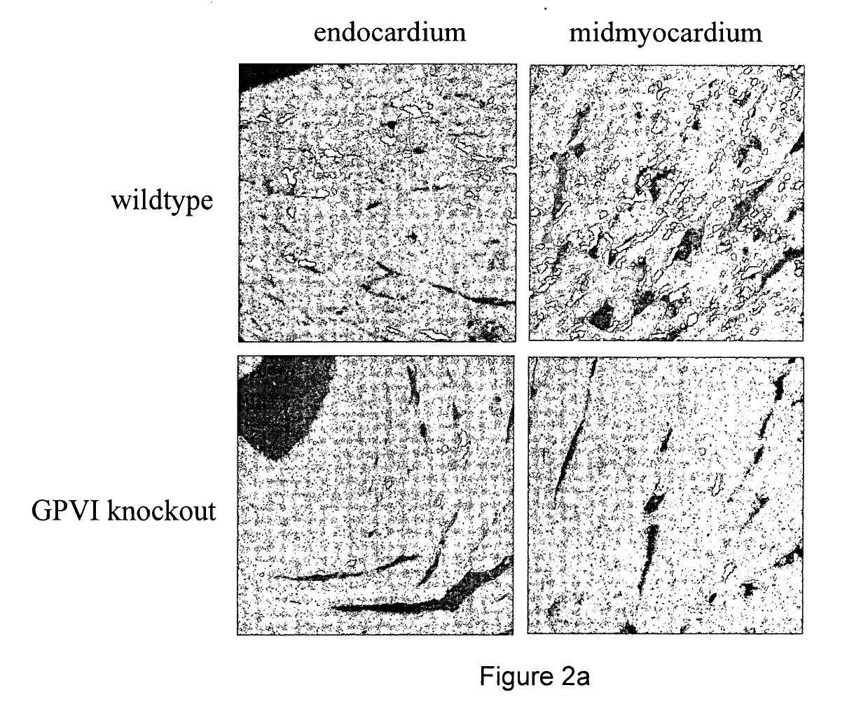

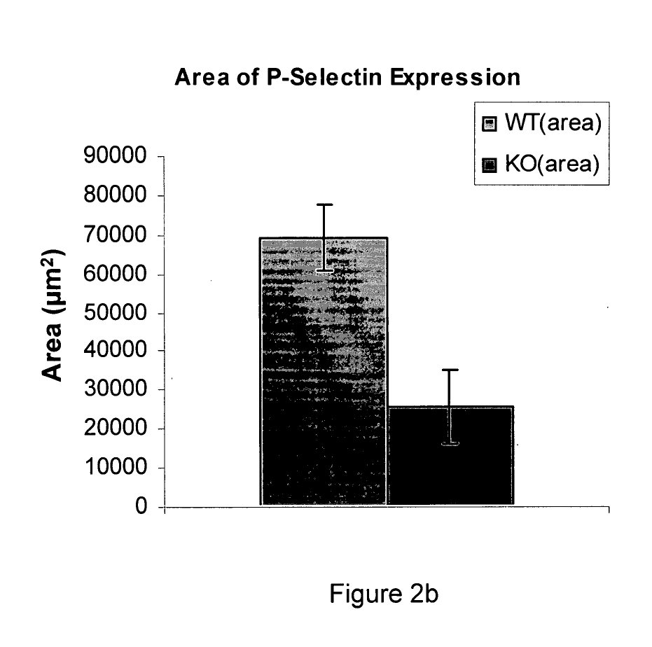

Fluorescent images: Fluorescent images were obtained using a Zeiss Confocal microscope (LSM510) or a conventional fluorescent microscope. Fluorescence was excited at 488 nm and detected at 540 nm. After 30 min ischemia and 15 min reperfusion, high levels of P-selectin were detected in the myocardium (endocardium and midmyocardium) of wild type mice (FIG. 2a). Much less P-selectin expression was detected in the myocardium of GPVI-knockout mice. To quantitate the level of expression, the size of the area with strong green fluorescence within the ischemic area was determined. The data showed that the total size of the area of P-selectin expression was significantly reduced in the hearts of GPVI-knockout mice as compared to wild type mice (FIG. 2b).

### Example 3

### Endothelial Reperfusion Injury

In a normal heart with healthy vasculature, a tight endothelium prevents collagen in the extracellular matrix of the vascular wall from contacting circulatory blood components. If the endothelium is damaged, such as during ischemia and reperfusion, collagen may become exposed. Because GPVI selectively binds to collagen, GPVI was used to investigate endothelial reperfusion injury in vivo. For this purpose, recombinant sGPVI was labeled with a fluorescent tag FITC (sGPVI-FITC) and sGPVI-FITC was injected intravenously into a mouse. The injected sGPVI-FITC binds to collagen that is exposed due to endothelial injury. The level of sGPVI-FITC binding to exposed collagen can be determined histologically under a fluorescent microscope and provides a measure for endothelial injury.

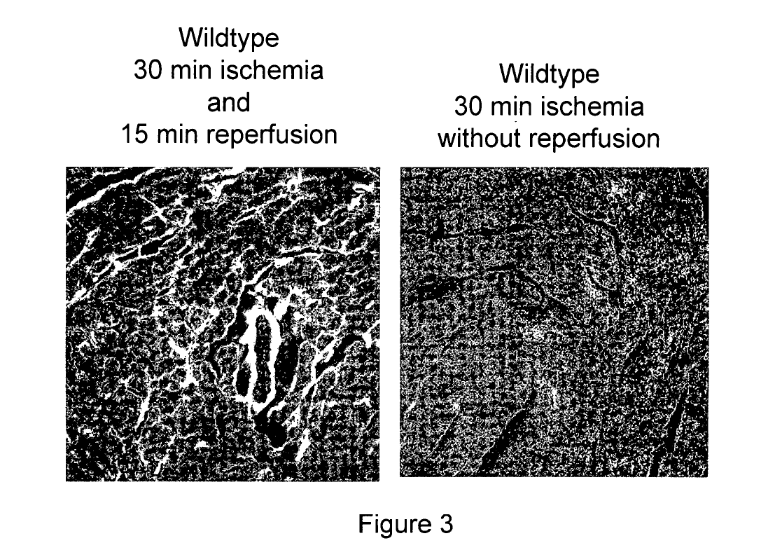

sGPVI-FITC (2 mg/kg) was injected into wild type mice 10 min prior to the onset of cardiac ischemia (30 min). In some animals ischemia was followed by reperfusion (15 min). In hearts that underwent reperfusion significant labeling of the vasculature was observed (FIG. 3), indicating significant injury of the endothelium and consequent exposure of collagen to circulatory blood components. In contrast, no labeling of the vasculature with sGPVI-FITC was observed in hearts that did not undergo reperfusion (FIG. 3). These data show that reperfusion of ischemic heart tissue results in endothelial injury.

### Example 4

### Cardioprotective Effect by an Anti-GPVI Antibody

Cynomolgus monkeys from China weighing 2.0-2.5 kg were used. A monkey selected for experimentation was fasted overnight and sedated with ketamine (10 mg/kg IM). Additionally, an injection of atropine (0.05 mg/kg IM) was given. An intravenous catheter was inserted into a leg vein. Anesthesia was achieved with sodium pentobarbital (10-15 mg/kg IV) and additional doses were administered throughout the experiment. Through a midline cervical incision the trachea was exposed, and an endotracheal tube was introduced. The animal was ventilated with the aid of a small animal respirator and a gas mixture of 40% O₂/60% N₂. A carotid artery was cannulated for measurement of blood pressure and collection of arterial blood samples. Then a left thoracotomy was performed in the fourth intercostal space and the heart was exposed. The left anterior descending coronary artery was occasionally visible, but was usually obscured by overlying fat. A 2-0 suture on a needle was blindly passed beneath the vascular bundle in the interventricular groove as close to the artery's origin as possible. The ends of the suture were passed through a short length of a polyethylene catheter to form a snare. Success of the snare was confirmed by observing cyanosis and cessation of contraction of the anterior wall of the heart when the snare was pulled for 10 sec, and then tissue hyperemia and resumption of contraction when the snare was released. A catheter was inserted into the left atrial appendage for microsphere injections. ECG leads were attached to measure heart rate and QRS morphology. A heating pad was used to warm the monkey to 38° C. measured rectally.

After completion of the surgical preparation and equilibration for at least 20 min, baseline heart rate, blood pressure and ECG were recorded, and the coronary artery was occluded for 90 min. ECG, heart rate, and blood pressure were continuously monitored and recorded every minute for 5 min, at 10 min, and then every 10 min until the end of the occlusion. At the end of the occlusion period, the snare was released and the coronary artery reperfused. Again ECG, heart rate, and blood pressure were recorded every minute for 5 min, at 10 min, and then every 10 min until the end of the 4-hour reperfusion period. If ventricular fibrillation developed, an electrical defibrillator was used in an attempt to convert the rhythm to sinus.

After 4 hrs of reperfusion, the heart was removed and hung by the aortic root on a perfusion apparatus. Saline was retrogradely perfused to wash blood from the coronary arteries and heart, and then 2-9 μm green fluorescent microspheres (Microgenics Corp., Freemont, Calif.) were added to the perfusate after reoccluding the coronary artery. Thus, the fluorescent microspheres entered only the myocardium perfused by patent coronary arteries and the risk area (or risk zone) was demarcated as the area of myocardium that did not contain fluorescent microspheres. The heart was placed on dry ice to freeze and then cut into 2-3 mm slices perpendicular to its long axis. The slices were incubated in 1% triphenyltetrazolium chloride (TTC) (GFS Chemicals, Powell, Ohio), warmed to 37° C. for 8-10 min and then put into 10% formalin for tissue preservation and enhancement of the color contrast between tissues stained and unstained by TTC. TTC stains normally perfused tissue with intact NADH stores brick red, whereas infarcted tissue in which this cofactor has been released and washed out is unstained and white or perhaps black from intramyocardial hemorrhage. Slices were compressed between plastic plates separated by exactly 2 mm. The size of risk zone regions identified under UV light and infarcted regions identified under white light were traced on plastic overlays. Areas were measured by planimetry and volumes calculated by multiplying the areas by 2 mm.

Control animals were subjected to coronary artery occlusion (90 min) and reperfusion (4 hrs) without administration of an anti-GPVI antibody. In one of the anti-GPVI antibody treated groups, the animals received a double dose of OM2 Fab fragment (a monoclonal mouse anti-human GPVI antibody; see Matsumoto et al., Thromb Res, 119:319-329, 2007; and U.S. Patent Application Publication No. 2007/0207155) (2 mg/kg each), the first dose administered 10 min prior to ischemia and the second dose administered just prior to reperfusion. Because the immunofluorescence data (see FIGS. 1-3) support a role of GPVI during reperfusion, in a second anti-GPVI antibody treated group, the animals received a single dose of OM2 Fab fragment (2 mg/kg) just prior to reperfusion. Cardiac infarction was analyzed by ANOVA, with a p value of <0.05 being considered statistically significant.

The infarct size was plotted against risk zone size rather than in a percentage graph. This is because the infarct size/risk zone size plot of the control animals does not go through the origin, as was also found for rodents (Ytrehus et al., Am J Physiol, 267:H2383-H2390, 1994). As Flameng et al. (Basic Res Cardiol 85:392-403, 1990) noted for baboons, risk zone size is also an important determinant of infarct size in macaques. Therefore, when the risk zone is small, infarct size will predictably be small even in the absence of any intervention. And when the risk zone is less than 0.6 cm³, no infarction is expected, even in control monkeys. When infarct size was plotted against risk zone size, OM2 antibody (either double or single dose) showed a significant cardioprotective effect as the regression lines shifted to the right. This shift shows that at the same risk zone size OM2-treated monkeys had a smaller infarct size. The extent of protection was similar in monkeys treated with double or single doses of OM2, consistent with the notion that platelet-collagen interaction via GPVI induces reperfusion injury and inhibition of such interaction provides cardioprotection.

To investigate whether OM2 inhibited platelet activation in these monkeys, blood samples were withdrawn before and after OM2 administration. Ex vivo collagen-induced platelet aggregation was then determined using a whole blood aggregometer (Chrono-log, Corporation, PA). Blood was diluted 1:1 (v/v) with saline and incubated at 37° C. for 5-10 min in the aggregometer before aggregation was initiated by collagen (0.5 μg/mL; Horm, Nycomed, Germany). Aggregation was monitored for 10 min as an increase of impedance of an electrode in the blood sample (Aggro/link v 4.75, Chrono-log).

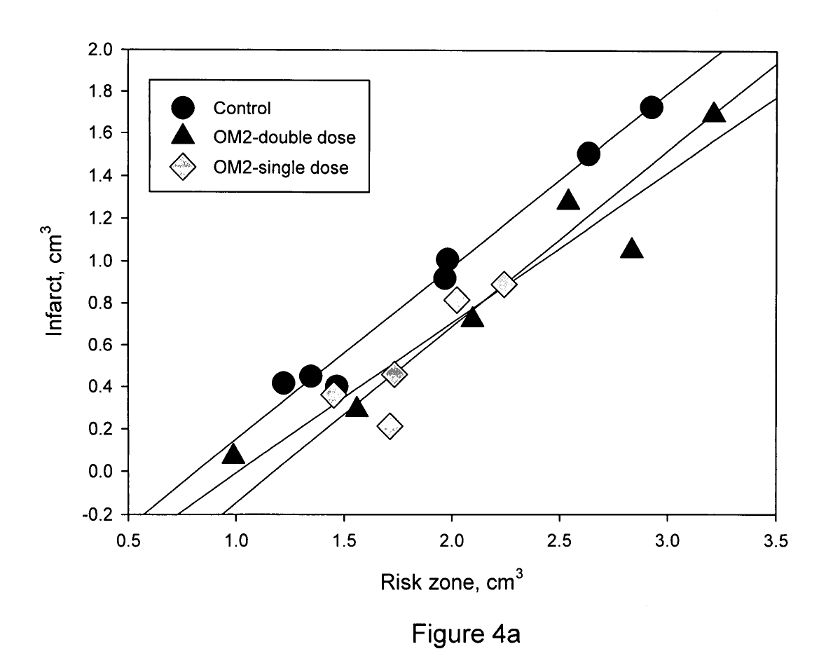

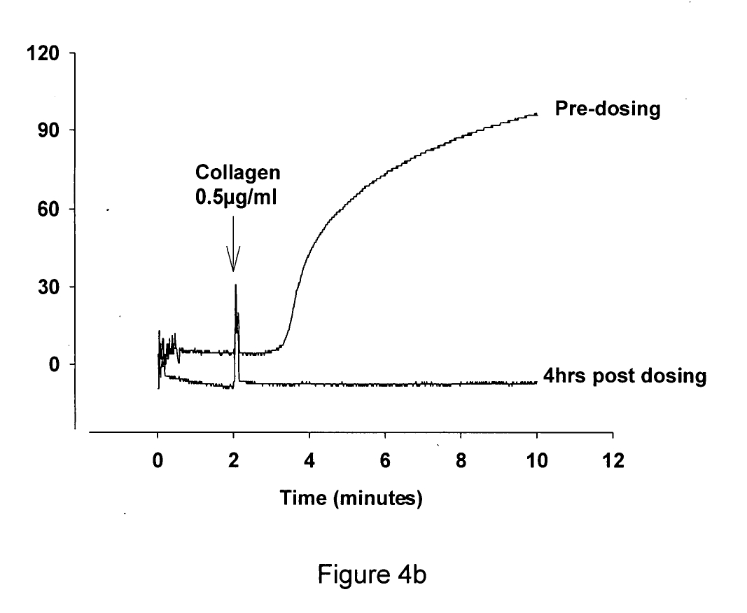

FIG. 4b shows representative measurements of collagen-induced platelet aggregation in blood samples taken before (pre-dosing) and 4 hrs after (4 hrs post-dosing) OM2 administration. The data demonstrate that OM2 (2 mg/kg) administered to the monkeys before reperfusion completely inhibited collagen-induced platelet aggregation, as measured in the ex vivo assay. OM2 administered at 0.4 mg/kg showed similar inhibition (data not shown).

The specification is most thoroughly understood in light of the teachings of the references cited within the specification, all of which are hereby incorporated by reference in their entirety. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for inhibiting reperfusion injury and/or infarction in a patient in need thereof comprising administering an inhibitor of platelet glycoprotein VI (GPVI), wherein said inhibitor inhibits the interaction between platelet GPVI and collagen.

2. The method of claim 1, wherein the reperfusion injury and/or infarction is a myocardial reperfusion injury and/or myocardial infarction.

3. The method of claim 1, wherein the reperfusion injury and/or infarction is an endothelial reperfusion injury and/or dysfunctional endothelium.

4. The method of claim 1, wherein the patient has had a heart attack.

5. The method of claim 1, wherein the patient requires an elective surgery resulting in a temporary blockage of coronary artery blood flow.

6. The method of claim 1, wherein the inhibitor is an antibody specific for a GPVI polypeptide, peptide, or naturally-occurring variant thereof.

7. The method of claim 6, wherein the antibody is selected from OM1, OM2, OM3, and OM4.

8. The method of claim 6, wherein the antibody is OM2.

9. The method of claim 6, wherein the antibody has been humanized.

10. The method of claim 6, wherein the antibody is an active antibody fragment selected from a chemically, enzymatically, or recombinantly produced Fab fragment, F(ab)₂ fragment, or peptide fragment comprising at least one complementarity determining region (CDR) specific for a GPVI polypeptide, peptide, or naturally-occurring variant thereof.

11. The method of claim 10, wherein the active antibody fragment is a fragment of an antibody selected from OM1, OM2, OM3, and OM4.

12. The method of claim 11, wherein the antibody is OM2.

13. The method of claim 10, wherein the active antibody fragment has been humanized.

14. The method of claim 1, wherein the inhibitor is a peptide fragment of GPVI.

15. The method of claim 14, wherein the peptide fragment of GPVI is a collagen-binding domain of GPVI.

16. The method of claim 14, wherein the peptide fragment is soluble GPVI (sGPVI).

17. Use of an inhibitor of platelet glycoprotein VI (GPVI) for the manufacture of a medicament for the treatment of reperfusion injury and/or infarction, wherein said inhibitor inhibits the interaction between platelet GPVI and collagen.

18. The use of claim 17, wherein the reperfusion injury and/or infarction is a myocardial reperfusion injury and/or myocardial infarction.

19. The use of claim 17, wherein the reperfusion injury and/or infarction is an endothelial reperfusion injury and/or dysfunctional endothelium.

20. The use of claim 17, wherein the inhibitor is an antibody specific for a GPVI polypeptide, peptide, or naturally-occurring variant thereof.

21. The use of claim 20, wherein the antibody is selected from OM1, OM2, OM3, and OM4.

22. The use of claim 21, wherein the antibody is OM2.

23. The use of claim 20, wherein the antibody has been humanized.

24. The use of claim 20, wherein the antibody is an active antibody fragment selected from a chemically, enzymatically, or recombinantly produced Fab fragment, F(ab)₂ fragment, or peptide fragment comprising at least one complementarity determining region (CDR) specific for a GPVI polypeptide, peptide, or naturally-occurring variant thereof.

25. The use of claim 24, wherein the active antibody fragment is a fragment of an antibody selected from OM1, OM2, OM3, and OM4.

26. The use of claim 25, wherein the antibody is OM2.

27. The use of claim 24, wherein the active antibody fragment has been humanized.

28. The use of claim 17, wherein the inhibitor is a peptide fragment of GPVI.

29. The use of claim 28, wherein the peptide fragment of GPVI is a collagen-binding domain of GPVI.

30. The use of claim 28, wherein the peptide fragment is soluble GPVI (sGPVI1).
